(19) United States
(12) Patent Application Publication
(10) Pub. No.: US 2025/0144340 A1
(43) Pub. Date: May 8, 2025

(54) WARNING MASK AND VIBRATION VEST WITH INHALE AND EXHALE COMMANDS

(71) Applicant: ATATÜRK ÜNIVERSITESI FIKRI MÜLKIYET HAKLARI KOORDINATÖRLÜGÜ DÖNER SERMAYE ISLETMESI, Yakutiye, Erzurum (TR)

(72) Inventors: Yasemin GÜLTEPE, Yakutiye, Erzurum (TR); Miraç Dilruba GEYIKOGLU, Yakutiye, Erzurum (TR); Mete YAGANOGLU, Yakutiye, Erzurum (TR); Faruk Baturalp GÜNAY, Yakutiye, Erzurum (TR); Gamze KOÇ, Yakutiye, Erzurum (TR)

(21) Appl. No.: 18/837,936

(22) PCT Filed: Dec. 27, 2023
(86) PCT No.: PCT/TR2023/051759

§ 371 Date: Aug. 13, 2024

(30) Foreign Application Priority Data
Dec. 30, 2022 (TR) 2022/021598

(51) Int. Cl. A61M 16/06; A61M 16/00

(52) U.S. Cl. CPC A61M 16/0616; A61M 16/0003; A61M 2205/3553; A61M 2205/50; A61M 2205/581

(57) ABSTRACT

Disclosed is a noninvasive mechanical ventilation (NIMV) treatment using an oronasal mask with continuous positive airway pressure (CRAP) and bilevel positive airway pressure (BiPAP) that by voice and vibrationally warns the patient to inhale and exhale at an adjustable time period instead of a person giving inhale-exhale command during the treatment process (approximately 2 hours) of patients diagnosed with hypercapnic respiratory failure (PCO₂ [partial carbon dioxide] level above 45 mmHg) who suffer from drowsiness with a decrease in consciousness levels, meets the above-mentioned requirements, eliminates all disadvantages and brings some additional advantages.

### TECHNICAL FIELD

The invention relates to a respirator system for noninvasive mechanical ventilation (NIMV) treatment using an oronasal mask with continuous positive airway pressure (CPAP) and bilevel positive airway pressure (BiPAP) that by voice and vibrationally warns the patient to inhale and exhale at an adjustable time period instead of a person giving inhale-exhale command during the treatment process (approximately 2hours) of patients diagnosed with hypercapnic respiratory failure (PCO₂ (partial carbon dioxide) level above 45 mmHg) who suffer from drowsiness with a decrease in consciousness levels.

### Prior Art

Arterial blood gas is a very important parameter in the evaluation of critical diseases. Blood gas analyses are an important procedure routinely performed to determine the severity of the condition and the etiology of the diseases in patients followed in the intensive care unit. Diagnoses of hypoxemic respiratory failure or hypercapnic respiratory failure are diagnoses made as a result of arterial blood gas analyzes. One of the most prominent symptoms in patients diagnosed with hypercapnic respiratory failure (PCO₂ (Partial Carbon Dioxide) levels exceeding 45 mmHg) is drowsiness that occurs with a decline in consciousness levels. The gold standard in treatment is noninvasive mechanical ventilation (NIMV). NIMV treatment is performed with CPAP and BiPAP modes available on the mechanical ventilator. An oronasal mask is also needed for treatment. This mask is placed on the patient's face, covering the mouth and nose. These patients, who are prone to sleep, cannot breathe in harmony with the mechanical ventilation device. For all these reasons, PCO₂ level in arterial blood gas reaches up to 90 mmHg and causes results up to continuation of the treatment by intubating the patient.

The most common reasons for failure to apply continuous positive airway pressure (CPAP) to cause mortality are the continuation or deepening of acidosis and an increase in partial carbon dioxide pressure. In order to prevent this, the patient who is connected to the mechanical ventilation device must also be accompanied by a person and be given inhale-exhale commands during the treatment (2 hours). This treatment method, which is applied to more than one person in the intensive care unit, should be evaluated in terms of causing ineffective labor force planning. Facing such a problem causes loss of workforce while trying to make effective labor force planning in intensive care units.

In the prior art, oronasal (covering the mouth and nose) mask is used. During application, the oxygen coming from the ventilator cannot be inhaled effectively while the patient is in apnea. For this reason, the patient's ventilation is performed by switching from Non-Invasive Mechanical Ventilation (NIMV) mode to Invasive Mechanical Ventilation (IMV) mode. This practice also causes nosocomial infections and as a result, an increase in the mortality rates can be observed. For this reason, the patient is constantly awakened by healthcare professionals and ventilation is tried to be ensured. As a result of ineffective manpower planning, labor force loss occurs.

When the studies in the current technique are examined, it is seen that there is a need for innovations in NIMV treatment, CPAP and BiPAP applications due to the limitations of the current technique in terms of patient health and comfort, employee comfort and human resources management.

### BRIEF DESCRIPTION OF THE INVENTION

In this description, the invention is described only for a better understanding of the subject matter and without any limiting effect.

The invention; relates to a respirator system for noninvasive mechanical ventilation (NIMV) treatment using an oronasal mask with continuous positive airway pressure (CPAP) and bilevel positive airway pressure (BiPAP) that by voice and vibrationally warns the patient to inhale and exhale at an adjustable time period instead of a person giving inhale-exhale command during the treatment process (approximately 2 hours) of patients diagnosed with hypercapnic respiratory failure (PCO₂ (partial carbon dioxide) level above 45 mmHg) who suffer from drowsiness with a decrease in consciousness levels.

The primary purpose of the invention is to warn the patient with a voiced warning system and vibration vest while the patient is in apnea and to ensure that a healthy ventilation process is automatically managed by the healthcare professional.

Another purpose of the invention is to reduce the invasive mechanical ventilation (IMV) rates of patients with hypercapnic respiratory failure.

Another purpose of the invention is to reduce the infection rates and mortality of patients with hypercapnic respiratory failure.

Another purpose of the invention is to provide a useful solution-oriented system that will help to achieve successful results in effective labor force planning.

The structural and characteristic features and all advantages of the invention will be understood more clearly thanks to the detailed description given below. Therefore, the evaluation should be made taking the detailed description into consideration.

### DETAILED DESCRIPTION OF THE INVENTION

In this detailed description, the inhale exhale commanded warning mask and vibration vest are described for a better understanding of the subject and in a way that does not have any limiting effect.

The invention is a respirator system that warns the patient to inhale and exhale in an adjustable time period by sound and vibration, instead of a person being with the patient and giving a inhale-exhale commands during the treatment process of patients with hypercapnic respiratory failure, in order to ensure that these patients who are prone to fall asleep due to drowsiness caused by a decrease in consciousness levels by using an oronasal mask with NIMV treatment CPAP and BiPAP to inhale and exhale in harmony with the mechanical ventilation device.

In its preferred form, the invention is a respirator system with voiced and vibrating warning feature that allows the patient to breathe in harmony with the mechanical ventilation device using an oronasal mask with CPAP and BiPAP during NIMV treatment.

In its preferred form, the invention is a mask and vest with a breathing warning command.

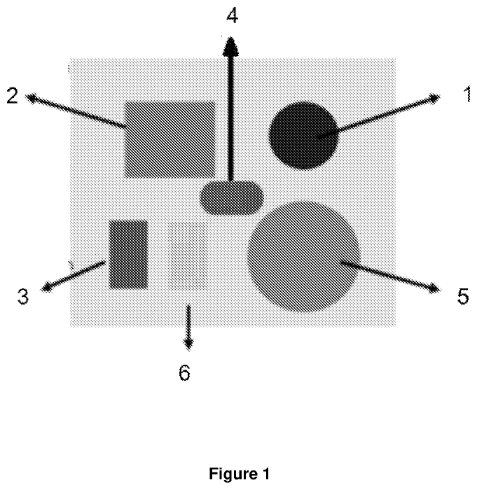

The system detailed below, which is given in FIG. 1, the warning mask with a breathing command and a vest of vibration consists of six parts.
i. Commutator (1): It is the key that the patient's ‘inhale’ and ‘exhale’ time is set by the treatment practitioner.
ii. Motion sensor that detects chest movement (2): It will detect the movement that occurs on the rib cage due to the breathing of the patient and transmit it to the microcontroller.
iii. Microcontroller (3): It evaluates the information received from the motion sensor (movement depending on breathing) within the framework of a certain algorithm (appropriate breathing time) and provides information to the person applying the treatment to direct the treatment of the patient.
iv. Voiced warning module (4): It gives the ‘inhale’, ‘exhale’ commands to the patient in a voiced manner in accordance with the time set by the treatment practitioner with the commutator.
v. Vibration motor (5): It allows the patient to be warned by vibration in addition to the ‘inhale’ and ‘exhale’ voice commands when the patient's breathing goes out of the warnings of the voiced warning module until it returns to normal.
vi. Communication module (6): It transmits the information received from the microcontroller mechanism to the treatment practitioner. It also helps the above devices on the patient to communicate within the system.

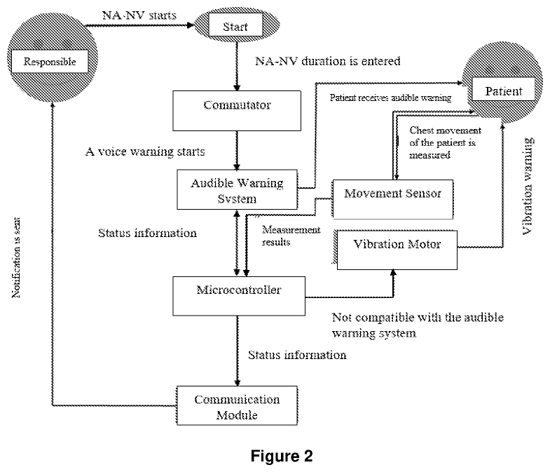

It is the working/workflow diagram of the system given in FIG. 2 and the working principle is; it initiates the inhale-exhale (In-Ex) time by setting the duration of the process during responsible NIMV treatment. The commutator sends a voiced warning to the patient in the form of In-Ex at the times set to the voiced warning system. The chest movement of the patient is taken through the vest and measured in the motion sensor and the measurement results are sent to the microcontroller.

With the warning mask and vibration vest with inhale and exhale commands with all these features, when the patient is in apnea in the field subject to the invention, the patient will be warned with a voiced warning system and vibration vest, and the process will be automatically managed by the healthcare worker, and patients with hypercapnic respiratory failure will have a decrease in invasive mechanical ventilation (IMV) and infection rates, a decrease in mortality will be provided and successful results in effective labor force planning will be obtained.

1. A system for allowing oronasal mask to enable sleep prone patients to breathe in harmony with the mechanical ventilation device, in patients with hypercapnic respiratory failure, continuous positive airway pressure (CPAP) and bilevel positive airway pressure (BiPAP) during noninvasive mechanical ventilation (NIMV) treatment with sleep state that occurs with decline in levels of consciousness, during the treatment (approximately 2 hours) instead of a person being with the patient and giving inhale-exhale commands, for providing the patients inhalation and exhalation process in an adjustable time period stimulated by sound and vibration, the system comprising:
a commutator,
a motion sensor that detects chest movement,
a microcontroller,
a voiced warning module,
a vibration motor, and
a communication module.

2. The system according to claim 1, wherein when the patient is in apnea the process can be managed automatically by the healthcare personnel by warning the patient with the voiced system and the vibrating vest.

3. The system according to claim 2, wherein the system can warn the patient by voice about breathing with the voiced warning system depending on the time instruction set by the health worker.

4. The system according to claim 1, wherein the system can measure the patient's chest movement through the sensor and send it to the microcontroller.

5. The system according to claim 1, wherein the system follows the compliance of the patient's chest movement measurement results with the voiced warning system, and in case of incompatibility, administers the vibration provided by the vibration motor to the patient with the vibration vest and continues the warning until the compliance of the chest movement measurement results with the voiced warning is ensured.

6. The system according to claim 1, wherein the system can instantly notify and warn the health worker about the compliance and/or incompatibility of the chest movement measurement results with the voiced warning system depending on the time instruction set by the health worker with the communication module.
